(19) United States
(12) Patent Application Publication
(10) Pub. No.: US 2009/0317804 A1
(43) Pub. Date: Dec. 24, 2009

(54) METHODS OF DETERMINING ANTIBIOTIC RESISTANCE

(75) Inventor: Adam M. BRISKA, Madison, WI (US)

Correspondence Address: COOLEY GODWARD KRONISH LLP;ATTN: Patent Group, Suite 1100, 777 - 6th Street, NW, WASHINGTON, DC, 20001, US

(73) Assignee: OPGEN INC., Madison, WI (US)

(21) Appl. No.: 12/120,592

(22) Filed: May 14, 2008

Related U.S. Application Data
(60) Provisional application No. 61/029,816, filed on Feb. 19, 2008.

(51) Int. Cl. C12Q 1/68

(52) U.S. Cl. 435/6

(57) ABSTRACT

This disclosure relates to methods of determining an antibiotic resistance profile of a bacterium, and methods of treating a patient with a therapeutically effective antibiotic. The methods include comparing the restriction map of the nucleic acid with a restriction map database, and determining antibiotic resistance of the bacterium by matching regions of the nucleic acid to corresponding regions in the database.

### RELATED APPLICATION

This application claims the benefit of U.S. provisional application Ser. No. 61/029,816 filed Feb. 19, 2008 in the U.S. Patent and Trademark office, which is hereby incorporated by reference herein in its entirety.

### TECHNICAL FIELD

This invention relates to methods of determining antibiotic resistance, methods of determining an antibiotic resistance profile of a bacterium, and methods of treating a patient with a therapeutically effective antibiotic.

### BACKGROUND

Bacteria and other microorganisms that cause infections are resilient and can develop ways to survive drugs meant to kill or weaken them, i.e., antibiotic resistance, antimicrobial resistance, or drug resistance. Several studies have demonstrated that patterns of antibiotic usage greatly affect the number of resistant organisms that develop. Other factors contributing towards resistance include incorrect diagnosis, unnecessary prescriptions, improper use of antibiotics by patients, and the use of antibiotics as livestock food additives for growth promotion.

Staphylococcus aureus is a prevalent antibiotic resistant pathogen. Overuse of broad-spectrum antibiotics, such as second- and third-generation cephalosporins, greatly hastens the development of Methicillin-resistant Staphylococcus aureus (MRSA), a very frequently identified antimicrobial drug-resistant pathogen found in U.S. hospitals. MRSA is sub-categorized as Community-Associated MRSA (CA-MRSA) or Hospital-Associated MRSA (HA-MRSA) depending upon the circumstances of infection by the bacterium, because the sub-categories represent distinct isolates of the bacterial species. MRSA isolates have evolved an ability to survive treatment with β-lactam antibiotics, for example penicillin, methicillin, and cephalosporins.

Unless antibiotic resistance problems are addressed, diseases that were previously treatable by administration of antibiotics will develop resistance to these previously effective drugs. There is a need for methods of determining antibiotic resistance, methods of determining an antibiotic resistance profile of a bacterium, and methods of treating a patient with a therapeutically effective antibiotic.

### SUMMARY

The present invention provides methods of determining antibiotic resistance. The methods include obtaining a restriction map of a nucleic acid from an organism and correlating the restriction map of the nucleic acid with a restriction map database, and determining antibiotic resistance of the bacterium by matching regions of the nucleic acid to corresponding regions in said database. With use of a detailed restriction map database, the organism can be identified and classified not just at a genus and species level, but also at a sub-species (strain), a sub-strain, and/or an isolate level. The featured methods offer fast, accurate, and detailed information for antibiotic resistance. The methods can be used in a clinical setting, e.g., a human or veterinary setting; or in an environmental or industrial setting (e.g., clinical or industrial microbiology, food safety testing, ground water testing, air testing, contamination testing, and the like). In essence, the invention is useful in any setting in which the detection and/or identification of antibiotic resistance of a microorganism is necessary or desirable.

In another aspect, the invention features a method of determining antibiotic resistance, the method including the steps of: (a) obtaining nucleic acid from a bacterium; (b) imaging the nucleic acid; (c) obtaining a restriction map of the nucleic acid; (d) comparing the restriction map of the nucleic acid with a restriction map database; and (e) determining antibiotic resistance of the bacterium by matching regions of the nucleic acid to corresponding regions in the database. In certain related embodiments, the method further includes the step of linearizing the nucleic acid.

In another aspect, the invention provides a method of determining antibiotic resistance profile of a bacterium, the method including the steps of: obtaining nucleic acid from a bacterium; preparing an optical map of the nucleic acid; identifying at least one motif present in the nucleic acid that is indicative of bacterial resistance; and correlating the at least one motif with resistance to one or more antibiotics, thereby to determine the antibiotic resistance profile for said bacterium. In a related embodiment of the method, the preparing step includes linearizing the nucleic acid, digesting the nucleic acid with one or more restriction enzymes, and labeling the nucleic acid for imaging.

In another aspect, the invention provides a method of determining a therapeutically effective antibiotic for treating a subject, the method including: (a) obtaining a sample from a patient, in which the sample is suspected to contain an infectious organism; (b) obtaining a nucleic acid from the organism; (b) preparing an optical map of said nucleic acid; (d) determining an antibiotic resistance profile of said organism by comparing said optical map with at least one database containing antibiotic resistance data; and (f) selecting a therapeutically effective antibiotic for treating said patient. In a related embodiment, the method further involves identifying the organism.

The detected organism can be a microorganism, a bacterium, a protist, a virus, a fungus, or disease-causing organisms including microorganisms such as protozoa and multicellular parasites. For example, the organism may be E. coli and S. aureus. In other embodiments, the organism is a community-acquired methicillin-resistant strain of S. aureus. Alternatively, the organism is a hospital-acquired methicillin-resistant strain of S. aureus.

The nucleic acid can be deoxyribonucleic acid (DNA), a ribonucleic acid (RNA) or can be a cDNA copy of an RNA obtained from a sample. The nucleic acid sample includes any tissue or body fluid sample (e.g., blood, sputum, saliva, urine, drainage from a body part, and drainage from an abscess), environmental sample (e.g., water, air, dirt, rock, plant material, etc.), and all samples prepared therefrom.

Methods of the invention can further include digesting nucleic acid with one or more enzymes, e.g., restriction endonucleases, e.g., BglII, NcoI, XbaI, and BamHI, prior to imaging. Preferred restriction enzymes include, but are not limited to:

| | | |
|---|---|---|
| AflII | ApaLI | BglII |
| AflII | BglII | NcoI |
| ApaLI | BglII | NdeI |
| AflII | BglII | MluI |
| AflII | BglII | PacI |
| AflII | MluI | NdeI |
| BglII | NcoI | NdeI |
| AflII | ApaLI | MluI |
| ApaLI | BglII | NcoI |
| AflII | ApaLI | BamHI |
| BglII | EcoRI | NcoI |
| BglII | NdeI | PacI |
| BglII | Bsu36I | NcoI |
| ApaLI | BglII | XbaI |
| ApaLI | MluI | NdeI |
| ApaLI | BamHI | NdeI |
| BglII | NcoI | XbaI |
| BglII | MluI | NcoI |
| BglII | NcoI | PacI |
| MluI | NcoI | NdeI |
| BamHI | NcoI | NdeI |
| BglII | PacI | XbaI |
| MluI | NdeI | PacI |
| Bsu36I | MluI | NcoI |
| ApaLI | BglII | NheI |
| BamHI | NdeI | PacI |
| BamHI | Bsu36I | NcoI |
| BglII | NcoI | PvuII |
| BglII | NcoI | NheI |
| BglII | NheI | PacI |

Imaging ideally includes labeling the nucleic acid. Labeling methods are known in the art and can include any known label. However, preferred labels are optically-detectable labels, such as 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron® Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; naphthalo cyanine, BOBO, POPO, YOYO, TOTO and JOJO.

A database for use in the invention can include a restriction map similarity cluster. The database can include a restriction map from at least one member of the clade of the organism. The database can include a restriction map from at least one subspecies of the organism. The database can include a restriction map from a genus, a species, a strain, a sub-strain, or an isolate of the organism. The database can include a restriction map with motifs common to a genus (e.g., a mec cassette), a species, a strain, a sub-strain, or an isolate of the organism.

Another database of the invention includes antibiotic resistance data. For example, the database includes resistance data of an organism to antibiotics such as methicillin, oxacillin, ciproflaxin, erythromycin, tetracyclin, clindamycin, trimethoprim/sulfa, vancomycin, penicillin G, levofloxacin, moxyfloxacin, rifampicin, linezolid, quinupristin/dalfopristin, gentamycin, and nitrofurantoin.

In one embodiment, a restriction map obtained from a single DNA molecule is compared against a database of restriction maps from known organisms having known antibiotic resistances in order to identify the closest match to a restriction fragment pattern occurring in the database. This process can be repeated iteratively until sufficient matches are obtained to identify an organism at a predetermined confidence level. According to methods of the invention, nucleic acid from a sample are prepared and imaged as described herein. A restriction map is prepared and the restriction pattern is correlated with a database of restriction patterns for known organisms. In a preferred embodiment, organisms are identified from a sample containing a mixture of organisms. In a highly-preferred embodiment, methods of the invention are used to determine a ratio of various organisms present in a sample suspected to contain more than one organism. Moreover, use of methods of the invention allows the detection of multiple microorganisms from the same sample, either serially or simultaneously.

In use, the invention can be applied to identify an antibiotic resistance profile of a microorganism making up a contaminant in an environmental sample. For example, methods of the invention are useful to identify a potentially antibiotic resistant biological hazard in a sample of air, water, soil, clothing, luggage, saliva, urine, blood, sputum, food, drink, and others. In a preferred embodiment, methods of the invention are used to detect and identify an antibiotic resistant profile in an organism in a sample obtained from an unknown source.

Further aspects and features of the invention will be apparent upon inspection of the following detailed description thereof.

All patents, patent applications, and references cited herein are incorporated in their entireties by reference.

### BRIEF DESCRIPTION OF THE DRAWINGS

FIG. 1 is a diagram showing restriction maps of six isolates of E. coli.

FIG. 2 is a diagram showing restriction maps of six isolates of E. coli clustered into three groups: 0157 (that includes O157:H7 and 536), CFT (that includes CFT073 and 1381), and K12 (that includes K12 and 718).

FIG. 3 is a diagram showing common motifs among restriction maps of six isolates of E. coli.

FIG. 4 is a diagram showing restriction maps of six isolates of E. coli, with the boxes indicating regions common to E. coli.

FIG. 5 is a diagram showing restriction maps of six isolates of E. coli, with the boxes indicating regions that are unique to a particular strain, namely 0157, CFT, or K12.

FIG. 6 is a diagram showing restriction maps of six isolates of E. coli, with the boxes indicating regions unique to each isolate.

FIG. 7 is a tree diagram, showing possible levels of identifying E. coli.

FIG. 8 is a diagram showing restriction maps of a sample (middle map) and related restriction maps from a database.

FIG. 9 is a clustering map showing genome similarity of isolates of S. aureus correlated with resistance of the isolates to various antibiotics.

### DETAILED DESCRIPTION

The present disclosure features methods of determining an antibiotic resistance profile of an organism, e.g., a microorganism. The methods include obtaining nucleic acid from a bacterium; imaging the nucleic acid; obtaining a restriction map of the nucleic acid, e.g., DNA, from an organism; comparing the restriction map of the nucleic acid with a restriction map database; and determining antibiotic resistance of the bacterium by matching regions of the nucleic acid to corresponding regions in the database.

Physical mapping of genomes, e.g., using restriction endonucleases to develop restriction maps, can provide accurate information about the nucleic acid sequences of various organisms. Restriction maps of, e.g., deoxyribonucleic acid (DNA), can be generated by optical mapping. Optical mapping can produce ordered restriction maps by using fluorescence microscopy to visualize restriction endonuclease cutting events on individual labeled DNA molecules.

With use of a detailed restriction map database that contains motifs common to various groups and sub-groups, the organism can be identified and classified not just at a genus and species level, but also at a sub-species (strain), a sub-strain, and/or an isolate level. For example, bacteria can be identified and classified at a genus level, e.g., Escherichia genus, species level, e.g., E. coli species, a strain level, e.g., 0157, CFT, and K12 strains of E. coli, and isolates, e.g., O157:H7 isolate of E. coli (as described in Experiment 3B below). The featured methods offer fast, accurate, and detailed information for antibiotic resistance of organisms. These methods can be used in a variety of clinical settings, e.g., for identification of an antibiotic resistant organism in a subject, e.g., a human or an animal subject.

Methods of the invention are also useful for identifying and/or detecting antibiotic resistance of an organism in food or in an environmental setting. For example, methods of the invention can be used to assess an environmental threat in drinking water, air, soil, and other environmental sources. Methods of the invention are also useful to identify organisms in food and to determine a common source of food poisoning in multiple samples that are separated in time or geographically, as well as samples that are from the same or similar batches.

This invention also features methods of determining antibiotic resistance, determining an antibiotic resistance profile of a bacterium, and determining a therapeutically effective antibiotic for treating a subject by, inter alia, determining antibiotic resistance of an organism via comparing the restriction map of the nucleic acid with a restriction map database; and determining antibiotic resistance of the bacterium by matching regions of the nucleic acid to corresponding regions in the database. These methods can be used in a clinical setting, e.g., human or veterinary setting.

Restriction Mapping

The methods featured herein utilize restriction mapping during both generation of the database and processing of an organism to determine an antibiotic resistance profile of the organism. One type of restriction mapping that can be used is optical mapping. Optical mapping is a single-molecule technique for production of ordered restriction maps from a single DNA molecule (Samad et al., Genome Res. 5:1-4, 1995). During this method, individual fluorescently labeled DNA molecules are elongated in a flow of agarose between a coverslip and a microscope slide (in the first-generation method) or fixed onto polylysine-treated glass surfaces (in a second-generation method). Id. The added endonuclease cuts the DNA at specific points, and the fragments are imaged. Id. Restriction maps can be constructed based on the number of fragments resulting from the digest. Id. Generally, the final map is an average of fragment sizes derived from similar molecules. Id. Thus, in one embodiment of the present methods, the restriction map of an organism to be identified is an average of a number of maps generated from the sample containing the organism.

Optical mapping and related methods are described in U.S. Pat. No. 5,405,519, U.S. Pat. No. 5,599,664, U.S. Pat. No. 6,150,089, U.S. Pat. No. 6,147,198, U.S. Pat. No. 5,720,928, U.S. Pat. No. 6,174,671, U.S. Pat. No. 6,294,136, U.S. Pat. No. 6,340,567, U.S. Pat. No. 6,448,012, U.S. Pat. No. 6,509,158, U.S. Pat. No. 6,610,256, and U.S. Pat. No. 6,713,263, each of which is incorporated by reference herein. Optical Maps are constructed as described in Reslewic et al., Appl Environ Microbiol. 2005 September; 71 (9):5511-22, incorporated by reference herein. Briefly, individual chromosomal fragments from test organisms are immobilized on derivatized glass by virtue of electrostatic interactions between the negatively-charged DNA and the positively-charged surface, digested with one or more restriction endonuclease, stained with an intercalating dye such as YOYO-1 (Invitrogen) and positioned onto an automated fluorescent microscope for image analysis. Since the chromosomal fragments are immobilized, the restriction fragments produced by digestion with the restriction endonuclease remain attached to the glass and can be visualized by fluorescence microscopy, after staining with the intercalating dye. The size of each restriction fragment in a chromosomal DNA molecule is measured using image analysis software and identical restriction fragment patterns in different molecules are used to assemble ordered restriction maps covering the entire chromosome.

Restriction Map Database

The database(s) used with the methods described herein can be generated by optical mapping techniques discussed supra. The database(s) can contain information for a large number of isolates, e.g., about 200, about 300, about 400, about 500, about 600, about 700, about 800, about 900, about 1,000, about 1,500, about 2,000, about 3,000, about 5,000, about 10,000 or more isolates. In addition, the restriction maps of the database contain annotated information (a similarity cluster) regarding motifs common to genus, species, sub-species (strain), sub-strain, and/or isolates for various organisms. The large number of the isolates and the information regarding specific motifs allows for accurate and rapid identification of an organism.

The restriction maps of the database(s) can be generated by digesting (cutting) nucleic acids from various isolates with specific restriction endonuclease enzymes. Some maps can be a result of digestion with one endonuclease. Some maps can be a result of a digest with a combination of endonucleases, e.g., two, three, four, five, six, seven, eight, nine, ten or more endonucleases. The exemplary endonucleases that can be used to generate restriction maps for the database(s) and/or the organism to be identified include: BglII, NcoI, XbaI, and BamHI. Non-exhaustive examples of other endonucleases that can be used include: AluI, ClaI, DpnI, EcoRI, HindIII, KpnI, PstI, SacI, and SmaI. Yet other restriction endonucleases are known in the art.

Map alignments between different strains are generated with a dynamic programming algorithm which finds the optimal alignment of two restriction maps according to a scoring model that incorporates fragment sizing errors, false and missing cuts, and missing small fragments (See Myers et al., Bull Math Biol 54:599-618 (1992); Tang et al., J Appl Probab 38:335-356 (2001); and Waterman et al., Nucleic Acids Res 12:237-242). For a given alignment, the score is proportional to the log of the length of the alignment, penalized by the differences between the two maps, such that longer, better-matching alignments will have higher scores.

To generate similarity clusters, each map is aligned against every other map. From these alignments, a pair-wise alignment analysis is performed to determine “percent dissimilarity” between the members of the pair by taking the total length of the unmatched regions in both genomes divided by the total size of both genomes. These dissimilarity measurements are used as inputs into the agglomerative clustering method “Agnes” as implemented in the statistical package “R”. Briefly, this clustering method works by initially placing each entry in its own cluster, then iteratively joining the two nearest clusters, where the distance between two clusters is the smallest dissimilarity between a point in one cluster and a point in the other cluster.

Organisms

Antibiotic resistance in various organisms, e.g., viruses, and various microorganisms, e.g., bacteria, protists, and fungi, can be identified with the methods featured herein. In one embodiment, the organism's genetic information is stored in the form of DNA. The genetic information can also be stored as RNA.

The sample containing the organism can be a human sample, e.g., a tissue sample, e.g., epithelial (e.g., skin), connective (e.g., blood and bone), muscle, and nervous tissue, or a secretion sample, e.g., saliva, urine, tears, and feces sample. The sample can also be a non-human sample, e.g., a horse, camel, llama, cow, sheep, goat, pig, dog, cat, weasel, rodent, bird, reptile, and insect sample. The sample can also be from a plant, water source, food, air, soil, plants, or other environmental or industrial sources.

Identifying Antibiotic Resistance in an Organism

The methods described herein, i.e., determining antibiotic resistance of an organism, determining an antibiotic resistance profile of a bacterium, and determining a therapeutically effective antibiotic to administer to a subject, include determining antibiotic resistance of an organism via comparing the restriction map of the nucleic acid with a restriction map database, and determining antibiotic resistance of the bacterium by matching regions of the nucleic acid to corresponding regions in the database. The methods involve comparing each of the raw single molecule maps from the unknown sample (or an average restriction map of the sample) against each of the entries in the database, and then combining match probabilities across different molecules to create an overall match probability.

In one embodiment of the methods, entire genome of the organism to be identified can be compared to the database. In another embodiment, several methods of extracting shared elements from the genome can be created to generate a reduced set of regions of the organism's genome that can still serve as a reference point for the matching algorithms, e.g., a Mec cassette.

As discussed above and in the Examples below, the restriction maps of the database can contain annotated information (a similarity cluster) regarding motifs common to genus, species, sub-species (strain), sub-strain, and/or isolates for various organisms. Such detailed information would allow identification of an organism at a sub-species level, which, in turn, would allow for a more accurate diagnosis and/or treatment of a subject carrying the organism.

In another embodiment, methods of the invention are used to identify genetic motifs that are indicative of an organism, strain, or condition. For example, methods of the invention are used to identify in an isolate at least one motif that confers antibiotic resistance. This allows appropriate choice of treatment without further cluster analysis.

Applications

The methods described herein can be used in a variety of settings, e.g., to determine antibiotic resistance in an organism in a human or a non-human subject, in food, in environmental sources (e.g., food, water, air), and in industrial settings. The featured methods also include determining a therapeutically effective antibiotic for treating a subject afflicted with a disease, e.g., a human or a non-human subject, and treating the subject based on the antibiotic resistance profile of the organism.

For example, Methicillin-resistant Staphylococcus aureus (MRSA) is a bacterium responsible for infections in humans. MRSA is sub-categorized as Community-Associated MRSA (CA-MRSA) or Hospital-Associated MRSA (HA-MRSA) depending upon the circumstances of infection by the bacterium, because the sub-categories represent distinct isolates of the bacterial species.

MRSA isolates have evolved an ability to survive treatment with β-lactam antibiotics, for example penicillin, methicillin, and cephalosporins. Without be limited by any theory or mechanism of action, resistance to methicillin has developed by acquisition of Mec genes, which code for an altered penicillin-binding protein (PBP) that has a lower affinity for binding β-lactams, for example, penicillins, cephalosporins and carbapenems. The Mec genes confer resistance to β-lactam antibiotics and obviates clinical use of these drugs for patients infected with MRSA isolates.

The methods of the invention herein can be used to determine whether a patient is infected with an isolate of S. aureus that contains an antibiotic resistance profile, and further whether the profile matches that of a CA-MRSA or a HA-MRSA (See Example 5). Once the resistance profile of the isolate is determined, a decision about treating the subject with a therapeutically effective antibiotic can be made, for example, by a medical provider. If the antibiotic resistance profile reveals that the isolate would be susceptible to methicillin, then the medical provider may treat the subject with methicillin. If the antibiotic resistance profile reveals that the isolate is resistant to methicillin, then the medical provider may treat the subject with a different antibiotic, for example, ciproflaxin, erythromycin, tetracyclin, clindamycin, trimethoprim/sulfa, vancomycin, penicillin G, levofloxacin, moxyfloxacin, rifampicin, linezolid, quinupristin/dalfopristin, gentamycin, or nitrofurantoin.

Other organisms in which an antibiotic resistance profile can be determined by the methods of the invention herein include Enterococcus faecium (a bacterium found in hospitals that is resistant to penicillin, vancomycin, and linezolid), Streptococcus pyogenes (a bacterium that is resistant to macrolide antibiotics), Streptococcus pneumoniae (a bacterium that is resistant to penicillin and other β-lactams), Proteus mirabilis (a bacterium that is sensitive to ampicillin and cephalosporins), Proteus vulgaris (a bacterium that is resistant to ampicillin and cephalosporins), Escherichia coli (a bacterium that is resistant to fluoroquinolone variants), Mycobacterium tuberculosis (a bacterium that is resistant to isoniazid and rifampin), and Pseudomonas aeruginosa (a bacteria that has shown low antibiotic susceptibility).

Other bacteria showing some antibiotic resistance include Salmonella, Campylobacter, and Streptococci, from each of which an antibiotic resistance profile can be determined by the methods of the invention herein.

The following examples provide illustrative embodiments of the present methods and should not be treated as restrictive.

### Example 1

### Microbial Identification Using Optical Mapping

Microbial identification (ID) generally has two phases. In the first, DNA from a number of organisms are mapped and compared against one another. From these comparisons, important phenotypes and taxonomy are linked with map features. In the second phase, single molecule restriction maps are compared against the database to find the best match.

Database Building and Annotation

Maps sufficient to represent a diversity of organisms, on the basis of which it will be possible to discriminate among various organisms, are generated. The greater the diversity in the organisms in the database, the more precise will be the ability to identify an unknown organism. Ideally, a database contains sequence maps of known organisms at the species and sub-species level for a sufficient variety of microorganisms so as to be useful in a medical or industrial context. However, the precise number of organisms that are mapped into any given database is determined at the convenience of the user based upon the desired use to which the database is to be put.

After sufficient number of microorganisms are mapped, a map similarity cluster is generated. First, trees of maps are generated. After the tree construction, various phenotypic and taxonomic data are overlaid, and regions of the maps that uniquely distinguish individual clades from the rest of the populations are identified. The goal is to find particular clades that correlate with phenotypes/taxonomies of interest, which will be driven in part through improvements to the clustering method.

Once the clusters and trees have been annotated, the annotation will be applied back down to the individual maps. Additionally, if needed, the database will be trimmed to include only key regions of discrimination, which may increase time performance.

Calling (Identifying) an Unknown

One embodiment of testing the unknowns involves comparing each of the raw single molecule maps from the unknown sample against each of the entries in the database, and then combining match probabilities across different molecules to create an overall match probability.

The discrimination among closely related organisms can be done by simply picking the most hits or the best match probability by comparing data obtained from the organism to data in the database. More precise comparisons can be done by having detailed annotations on each genome for what is a discriminating characteristic of that particular genome versus what is a common motif shared among several isolates of the same species. Thus, when match scores are aggregated, the level of categorization (rather than a single genome) will receive a probability. Therefore, extensive annotation of the genomes in terms of what is a defining characteristic and what is shared will be required.

In one embodiment of the method, entire genomes will be compared to all molecules. Because there will generally be much overlap of maps within a species, another embodiment can be used. In the second embodiment, several methods of extracting shared elements from the genome will be created to generate a reduced set of regions that can still serve as a reference point for the matching algorithms. The second embodiment will allow for streamlining the reference database to increase system performance.

### Example 2

### Using Multiple Enzymes for Microbial Identification

In one embodiment, the single molecule restriction maps from each of the enzymes will be compared against the database described in Example 1 independently, and a probable identification will be called from each enzyme independently. Then, the final match probabilities will be combined as independent experiments. This embodiment will provide some built-in redundancy and therefore accuracy for the process.

### INTRODUCTION

In general, optical mapping can be used within a specific range of average fragment sizes, and for any given enzyme there is considerable variation in the average fragment size across different genomes. For these reasons, it typically will not be optimal to select a single enzyme for identification of clinically-relevant microbes. Instead, a small set of enzymes will be chosen to optimize the probability that for every organism of interest, there will be at least one enzyme in the database suitable for mapping.

Selection Criteria

A first step in the selection of enzymes was the identification of the bacteria of interest. These bacteria were classified into two groups: (a) the most common clinically interesting organisms and (b) other bacteria involved in human health. The chosen set of enzymes must have at least one enzyme that cuts each of the common clinically interesting bacteria within the range of average fragment sizes suitable for detailed comparisons of closely related genomes (about 6-13 kb). Additionally, for the remaining organisms, each fragment must be within the functional range for optical mapping (about 4-20 kb). These limits were determined through mathematical modeling, directed experiments, and experience with customer orders. Finally, enzymes that have already been used for Optical Mapping were selected.

Suggested Set

Based upon the above criteria, the preliminary set consisted of the enzymes BglII, NcoI, and XbaI, which have been used for optical mapping. There are 28 additional sets that cover the key organisms with known enzymes, so in the event that this set is not adequate, there alternatives will be utilized (data not shown).

Final Steps

Because the analysis in Experiment 2 is focused on the sequenced genomes, prior to full database production, this set of enzymes will be tested against other clinically important genomes, which will be part of the first phase of the proof of principle study.

### Example 3

### Identification of E. coli

In one embodiment of a microbial identification method, nucleic acids of between about 500 and about 1,000 isolates will be optically mapped. Then, unique motifs will be identified across genus, species, strains, substrains, and isolates. To identify a sample, single nucleic acid molecules of the sample will be aligned against the motifs, and p-values assigned for each motif match. The p-values will be combined to find likelihood of motifs. The most specific motif will give the identification.

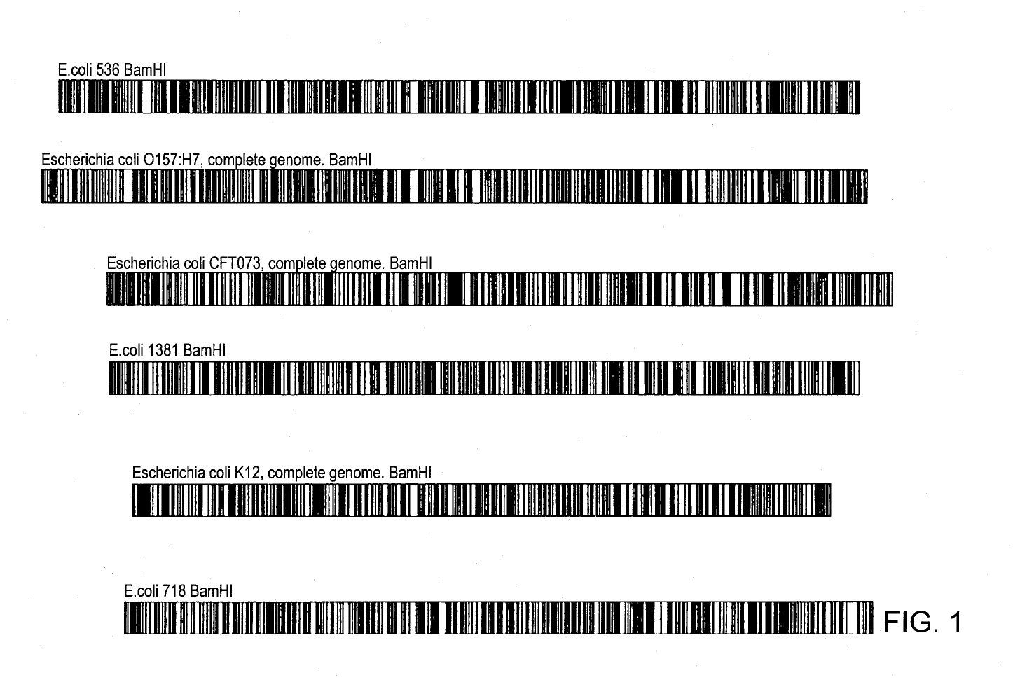

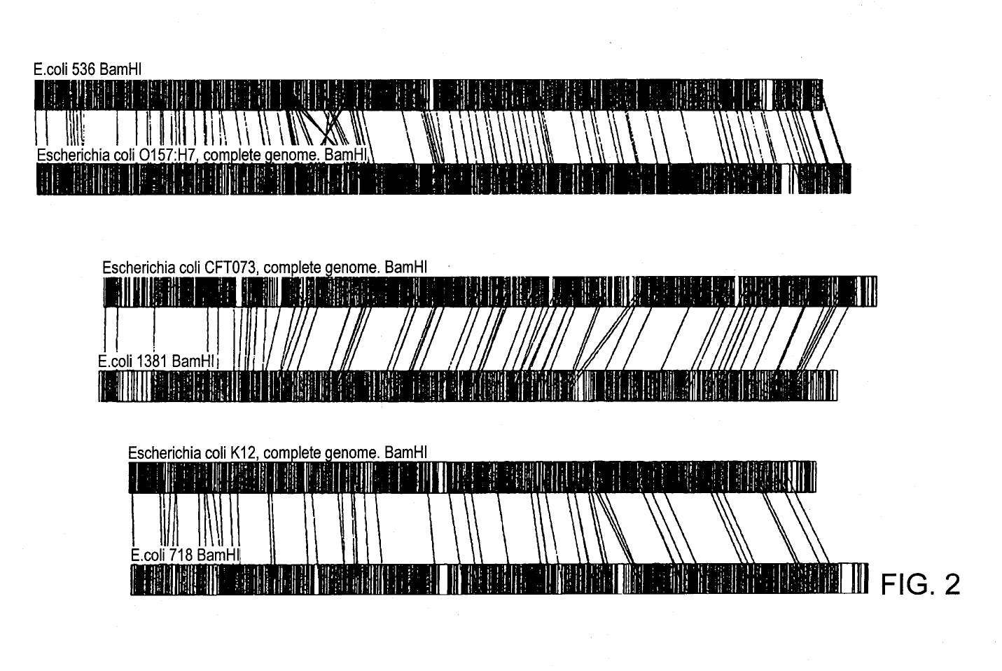

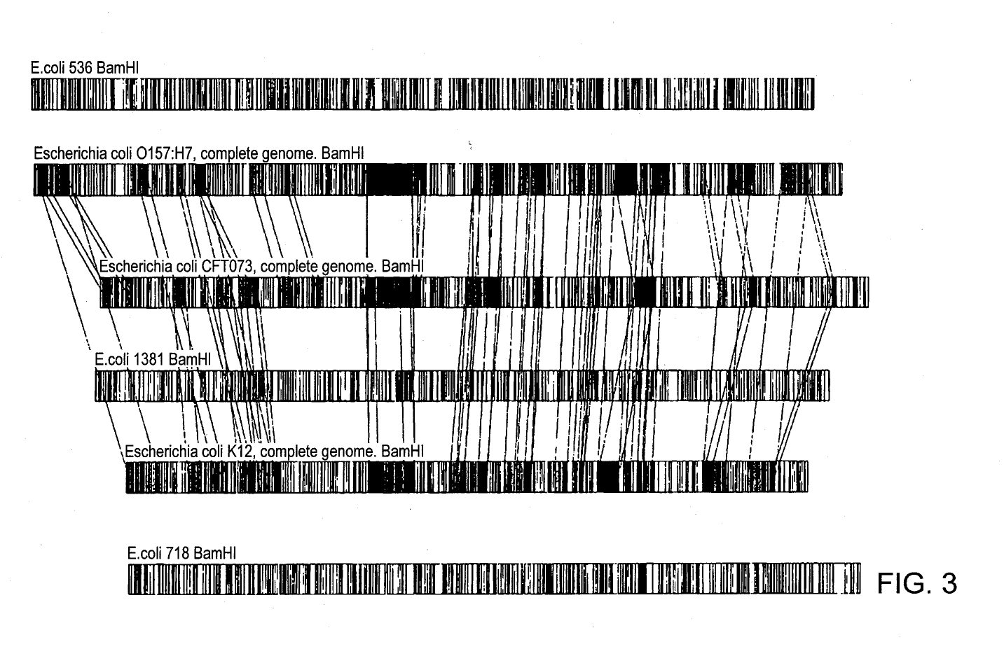

The following embodiment illustrates a method of identifying E. coli down to an isolate level. Restriction maps of six E. coli isolates were obtained by digesting nucleic acids of these isolates with BamHI restriction enzyme. FIG. 1 shows restriction maps of these six E. coli isolates: 536, O157:H7 (complete genome), CFT073 (complete genome), 1381, K12 (complete genome), and 718. As shown in FIG. 2, the isolates clustered into three sub-groups (strains): 0157 (that includes O157:H7 and 536), CFT (that includes CFT073 and 1381), and K12 (that includes K12 and 718).

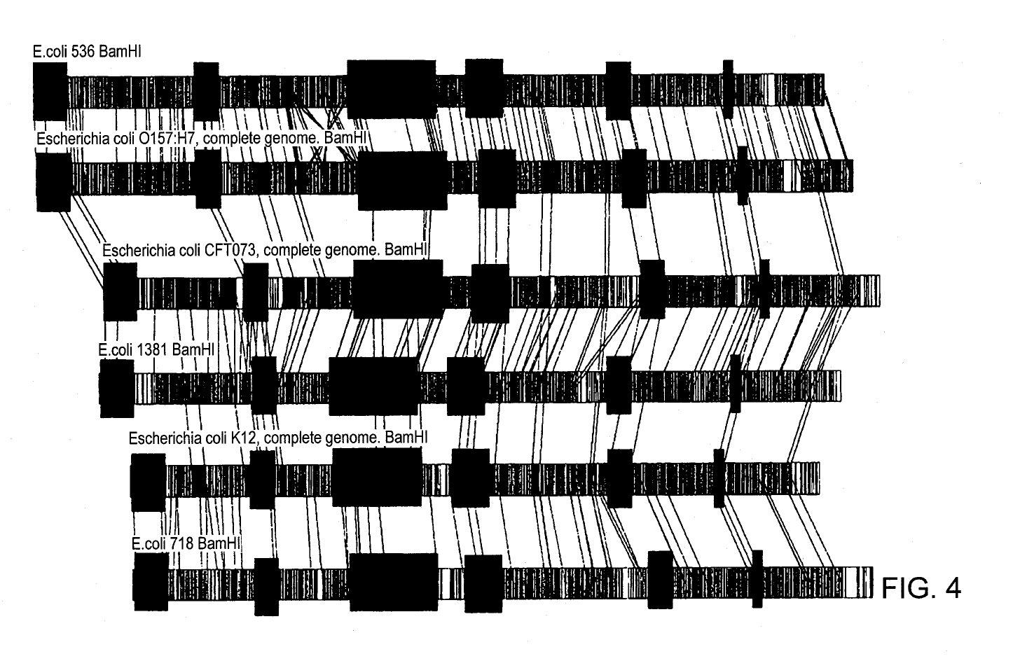

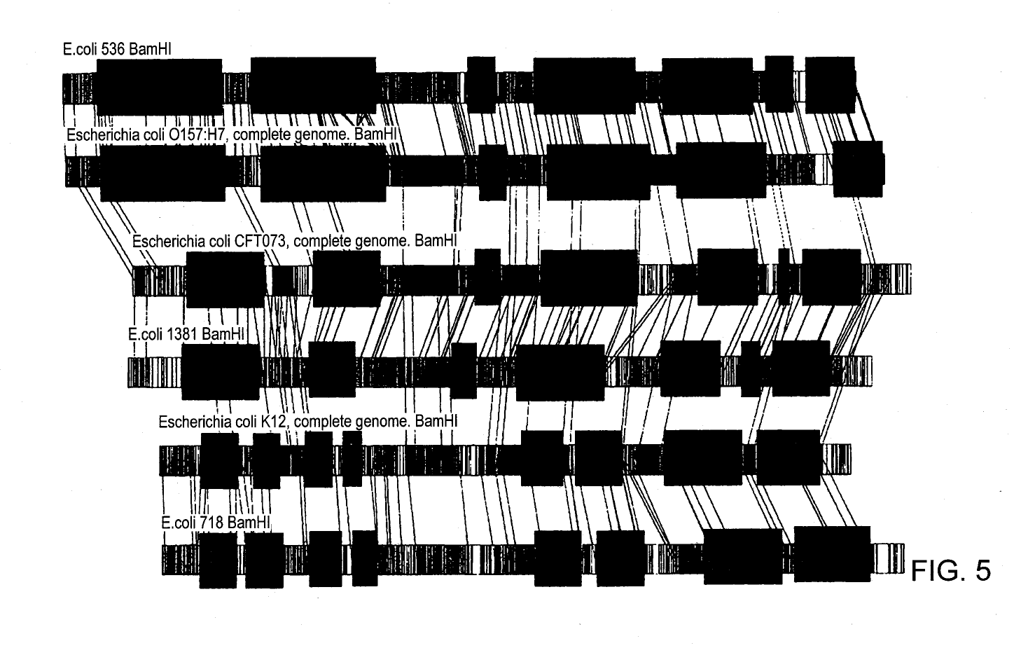

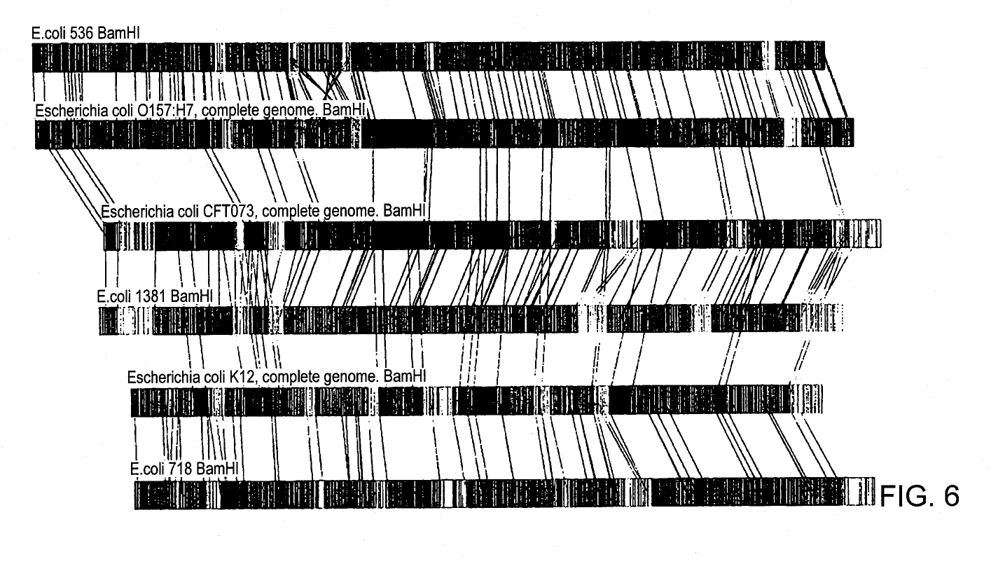

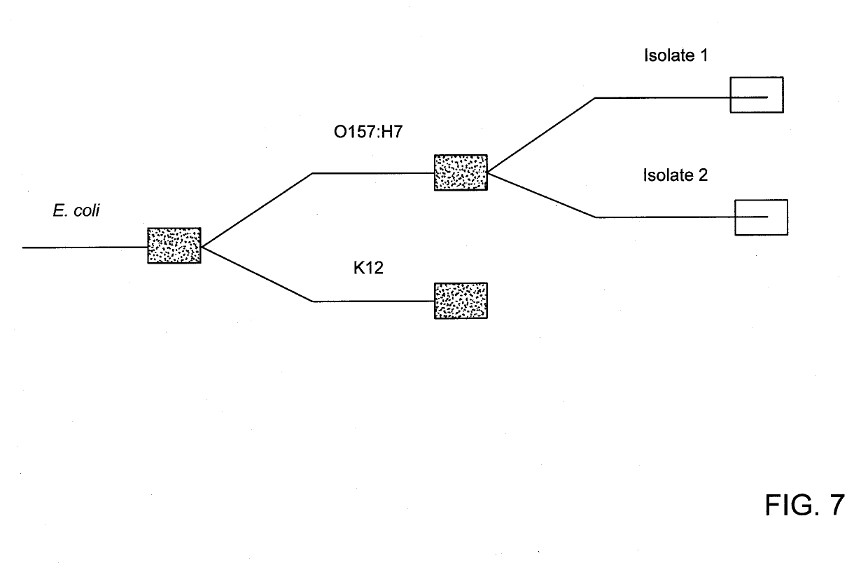

These restriction maps provided multi-level information regarding relation of these six isolates, e.g., showed motifs that are common to all of the three sub-groups (see, FIG. 3) and regions specific to E. coli (see, boxed areas in FIG. 4). The maps were also able to show regions unique to each strain (see, boxed areas in FIG. 5) and regions specific to each isolate (see boxed regions in FIG. 6).

This and similar information can be stored in a database and used to identify bacteria of interest. For example, a restriction map of an organism to be identified can be obtained by digesting the nucleic acid of the organism with BamHI. This restriction map can be compared with the maps in the database. If the map of the organism to be identified contains motifs specific to E. coli, to one of the sub-groups, to one of the strains, and/or to a specific isolate, the identity of the organism can be obtained by correlating the specific motifs. FIG. 6 shows a diagram to illustrate the possibilities of traversing variable lengths of a similarity tree.

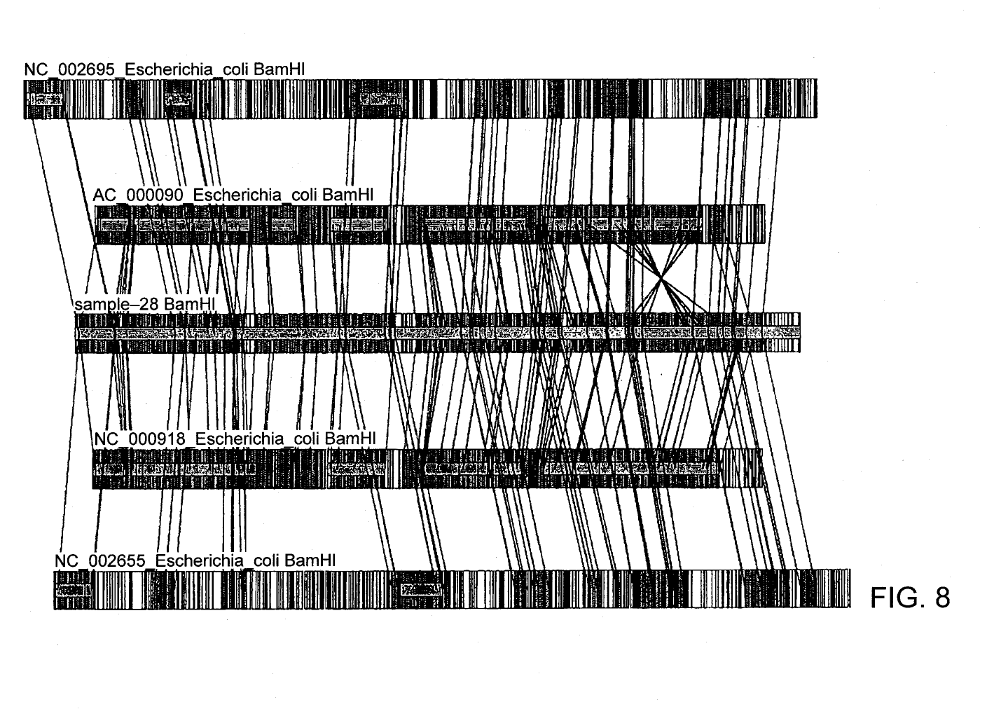

The following example illustrates identifying a sample as an E. coli bacterium. A sample (sample 28) was digested with BamHI and its restriction map obtained (see FIG. 8, middle restriction map). This sample was aligned against a database that contained various E. coli isolates. The sample was found to be similar to four E. coli isolates: NC 002695, AC 000091, NC 000913, and NC 002655. The sample was therefore identified as E. coli bacterium that is most closely related to the AC 000091 isolate.

### Example 4

### Genomic Differences in Clinical Isolates of Methicillin-Resistant S. aureus

Staphylococcus aureus is a major nosocomial and community-acquired pathogen with a rapidly evolving genome that presents an important clinical challenge. Data herein show that the complete genome sequence from several staphylococcal isolates indicated that many of the genes involved in virulence are primarily carried on mobile genetic islands (GIs).

Optical mapping was used to characterize whole genome maps of five unsequenced Methicillin-Resistant Staphylococcus aureus (MRSA) isolates (Wisconsin strains WI-23, WI-33, WI-34, WI-99, WI-591). The genome maps of the Wisconsin strains were then compared to five sequenced genomes (N315, MW2, COL, Mu50, USA300-FPR3757). Data herein show a variety of genomic differences that were identified in the unsequenced isolates including the presence of unique genomic islands. Map data show that most of the Wisconsin strains cluster with the sequenced strain MW2 while strain WI-23 appeared outside of this cluster due to the presence of five unique genomic islands.

Optical Mapping

Optical Maps were constructed for MRSA isolates WI-23, WI-33, WI-99, WI-591, USA300-114, USA300-FPR3757 and MW2 according to Reslewic et al. (Appl Environ Microbiol. 2005 September; 71 (9):5511-22). Briefly, high molecular weight DNA molecules from each isolate (Sanjay Shukla, Marshfield Clinic Research Foundation) were immobilized as individual molecules onto Optical Chips. The immobilized molecules were digested with a restriction enzyme, XbaI (NEB), fluorescently stained with YOYO-1 (Invitrogen), and positioned onto an automated fluorescent microscope for image capture and single molecule markup (Pathfinder). Software was used to record size and order of restriction fragments for each molecule, resulting in high-resolution single molecule restriction maps. Collections of single-molecule restriction maps were then assembled (Gentig) according to overlapping fragment patterns to produce whole genome, ordered restriction maps.

Map Alignments

Map alignments between different strains were generated using a dynamic programming algorithm that determines the optimal alignment of two restriction maps according to a scoring model that incorporates fragment sizing errors, false and missing cuts, and missing small fragments (See Myers et al., Bull Math Biol 54:599-618 (1992); Tang et al., J Appl Probab 38:335-356 (2001); and Waterman et al., Nucleic Acids Res 12:237-242). For a given alignment, the score is proportional to the log of the length of the alignment, penalized by the differences between the two maps, such that longer, better-matching alignments will have higher scores.

Map Clustering

To generate similarity clusters, each maps were aligned to the other maps. From these alignments, a pair wise percent dissimilarity was calculated by taking the total length of the unmatched regions in both genomes divided by the total size of both genomes. These dissimilarity measurements were used as inputs into the agglomerative clustering method agnes as implemented in the statistical package “R”. This clustering method works by initially placing each entry in its own cluster, then iteratively joining the two nearest clusters, such that the distance between two clusters is the smallest dissimilarity between a point in one cluster and a point in another cluster.

Results

An Optical Map was prepared from the sequenced strain MW2 and then compared directly to the corresponding in silico (derived from sequence) restriction map. The Optical Map genome size was determined to be 2,798,991 bp compared to 2,822,174 bp calculated from sequence, a difference of 0.82% underestimate in the Optical Map. Fragments matched within 2% sizing and the relative ordering of fragments between the two maps was entirely consistent.

Optical Maps were also prepared from the unsequenced S. aureus strains WI-23, WI-33, WI-34, WI-99, WI-591 and USA300-0114, and then compared with the in-silico restriction maps of the sequenced S. aureus strains N315, Mu50, MW2, COL, and USA300-FPR3757. Whole-genome, map-based clustering was then performed. Four of the Wisconsin isolates (WI-99, WI-34, WI-33, and WI-591) clustered very closely with the typical community-acquired MRSA MW2 (USA 400). Further analysis using map-based pairwise comparisons pinpointed a novel 42 kb insertion in WI-33 relative to MW2.

The MW2 phage harboring the Panton-Valentine leucocidin (PVL)-encoding genes (prevalent among CA-MRSA strains) was absent in the WI-591 isolate whereas WI-99, WI-34 and WI-33 appeared to contain this island. No significant map-based differences were identified in the WI-99 and WI-34 isolates when compared with MW2.

Map-based genomic clustering indicated that the WI-23 isolate was more dissimilar than the other Wisconsin isolates studied. Further inspection by pairwise comparison between this isolate and the five sequenced strains indicated WI-23 contained five genomic islands, totaling 257 Kb, unique to this isolate.

### CONCLUSION

Data above show that CA-MRSA strains WI-33, WI-34, WI-99, and WI-591 were very similar in gene content to the sequenced strain MW2. The data further show that CA-MRSA strain WI-33 contains a novel 42 Kb genomic island, and that CA-MRSA strain WI-23 contains five novel genomic islands, totaling 257 Kb.

### Example 5

### Methicillin Resistance Profile of Isolates of S. aureus

Methicillin resistance and presence of the PVL toxin gene (responsible for many of the severe clinical symptoms of infection with methicillin-resistant S. aureus (MRSA), such as furunculosis, severe necrotizing pneumonia, and necrotic lesions of the skin and soft tissues) are two clinically-important phenotypic traits. To identify a methicillin resistance profile, 69 different isolates of S. aureus were analyzed.

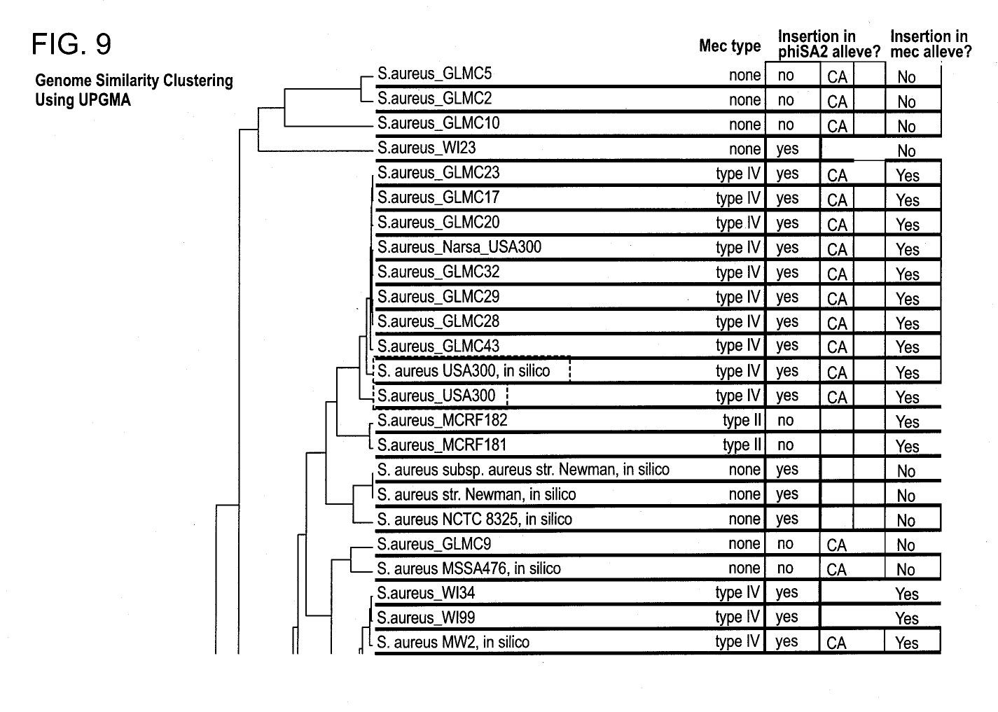

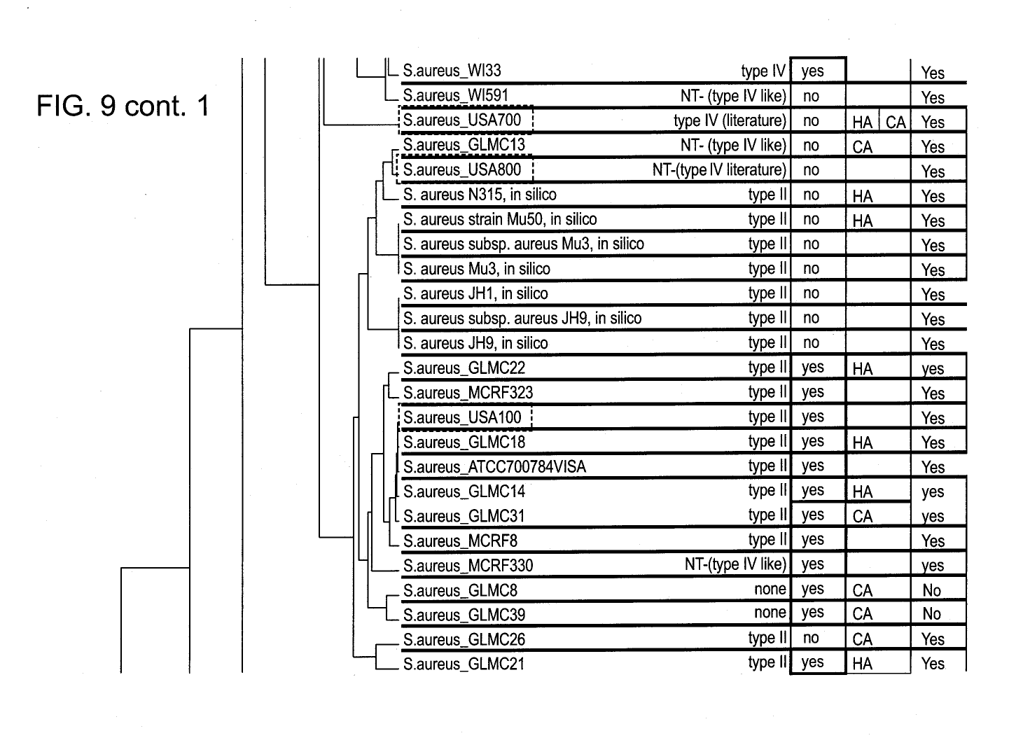

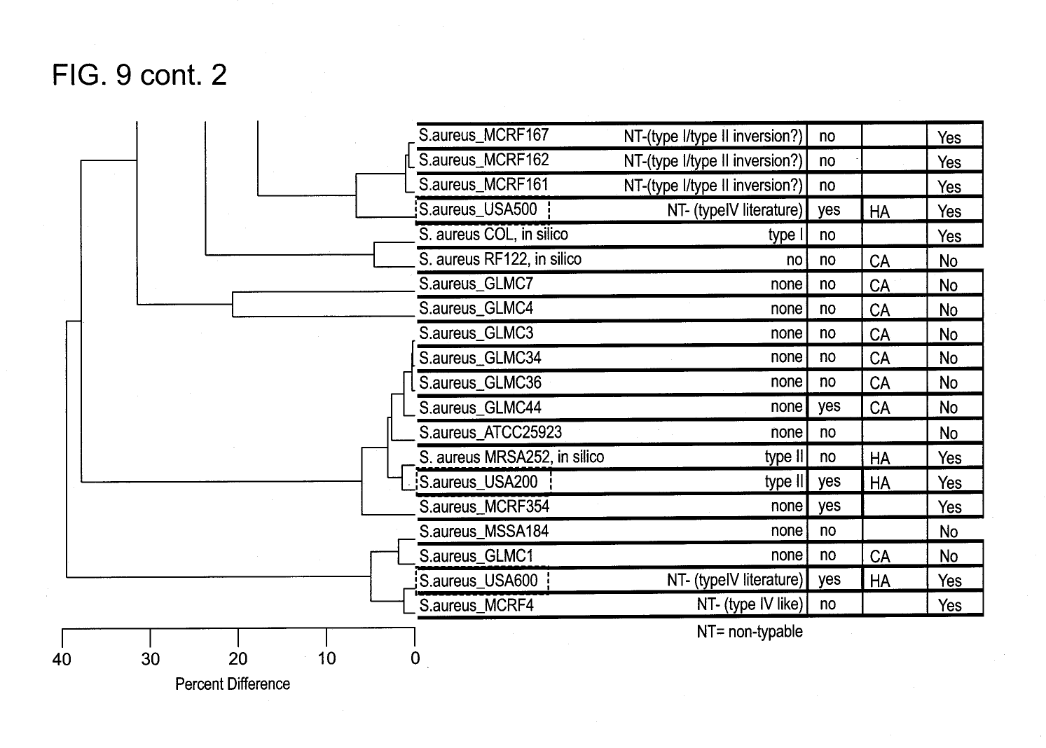

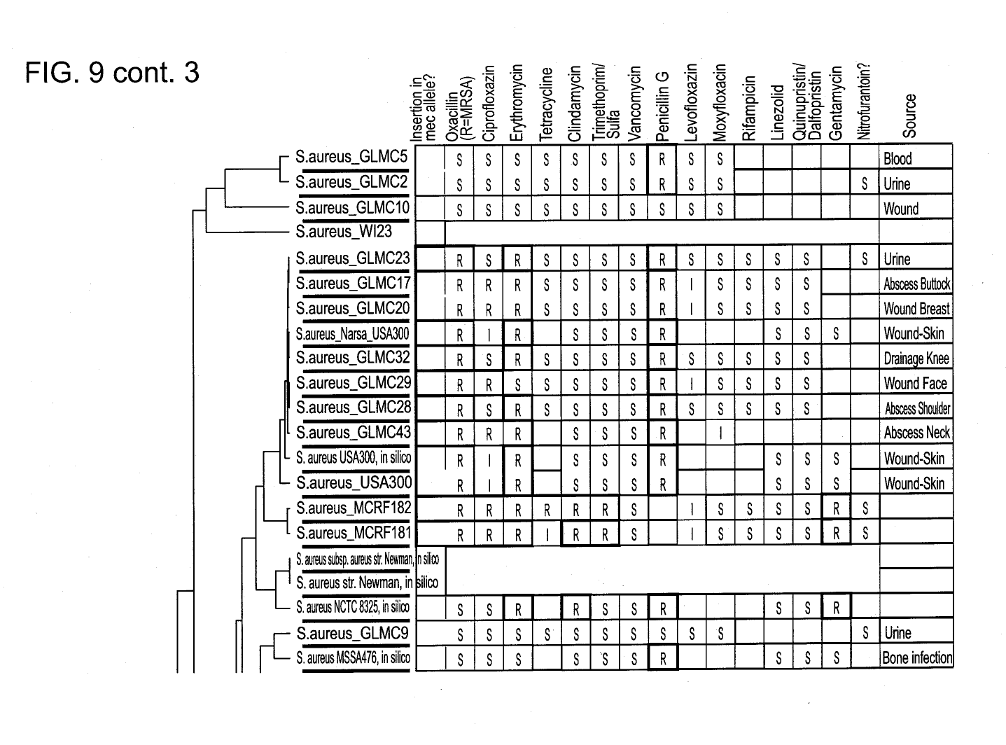

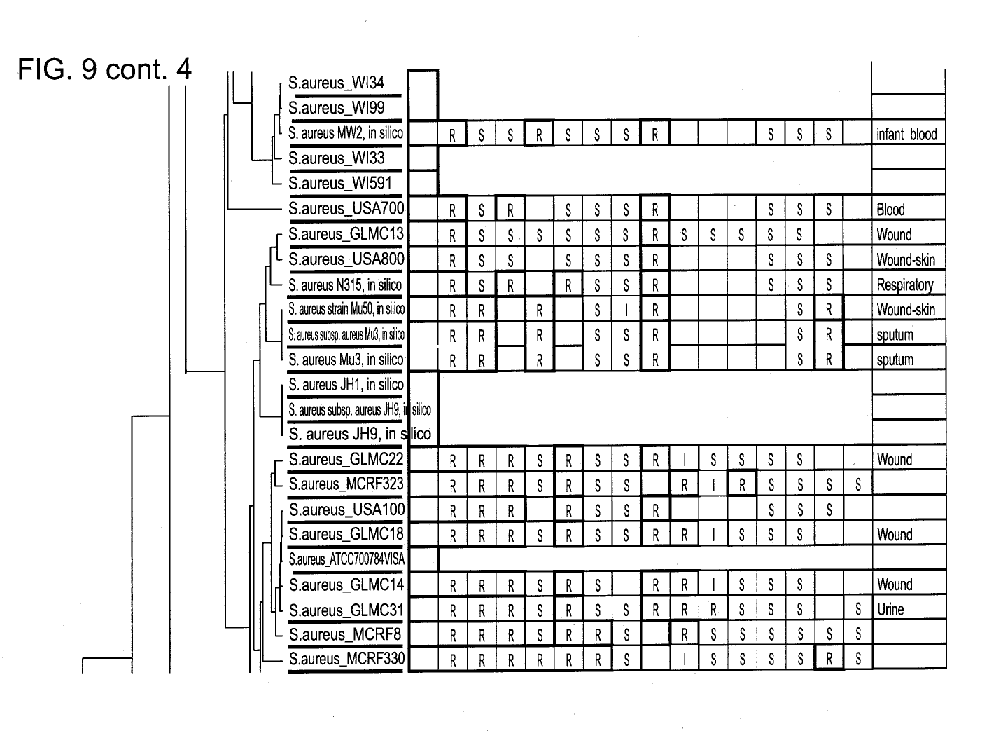

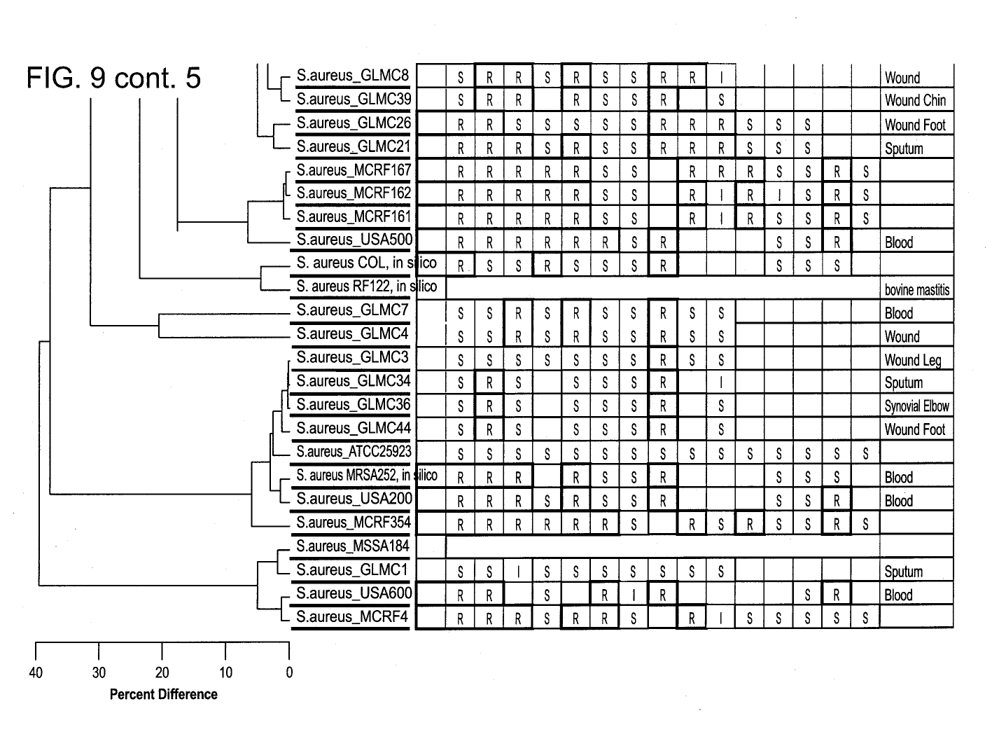

Optical mapping was used to characterize genome maps of 69 isolates of S. aureus. The genome maps of the isolates were assessed for insertion of additional DNA at the insertion sites for Mec genes and PVL toxin genes. Data herein show a correlation between the presence of motifs inserted at both loci. and methicillin resistance (FIG. 9).

Optical Mapping

Optical Maps were constructed for the 69 isolates according to Reslewic et al. (Appl Environ Microbiol. 2005 September; 71 (9):5511-22). Briefly, high molecular weight DNA molecules from each isolate (Sanjay Shukla, Marshfield Clinic Research Foundation) were immobilized as individual molecules onto Optical Chips. The immobilized molecules were digested with a restriction enzyme, XbaI (NEB), fluorescently stained with YOYO-1 (Invitrogen), and positioned onto an automated fluorescent microscope for image capture and single molecule markup (Pathfinder). Software was used to record size and order of restriction fragments for each molecule, resulting in high-resolution single molecule restriction maps. Collections of single-molecule restriction maps were then assembled (Gentig) according to overlapping fragment patterns to produce ordered restriction maps.

Map Alignments

Map alignments between the different isolates were generated using a dynamic programming algorithm that determines the optimal alignment of two restriction maps according to a scoring model that incorporates fragment sizing errors, false and missing cuts, and missing small fragments (See Myers et al., Bull Math Biol 54:599-618 (1992); Tang et al., J Appl Probab 38:335-356 (2001); and Waterman et al., Nucleic Acids Res 12:237-242). For a given alignment, the score is proportional to the log of the length of the alignment, penalized by the differences between the two maps, such that longer, better-matching alignments will have higher scores.

Map Clustering

To generate similarity clusters, each maps were aligned to the other maps. From these alignments, a pair wise percent dissimilarity was calculated by taking the total length of the unmatched regions in both genomes divided by the total size of both genomes. These dissimilarity measurements were used as inputs into the agglomerative clustering method agnes as implemented in the statistical package “R”. This clustering method works by initially placing each entry in its own cluster, then iteratively joining the two nearest clusters, such that the distance between two clusters is the smallest dissimilarity between a point in one cluster and a point in another cluster.

Results

Data herein show clustering of S. aureus strains by map similarity and that an antibiotic resistance phenotype tends to cluster (FIG. 9). Based on the map data, a correlation between the presence of motifs inserted at both loci. and methicillin resistance was observed, i.e., an isolate of S. aureus that was found to have an insertion of additional DNA at the insertion site of the Mec gene was also found to have a resistance to methicillin (FIG. 9).

Thus methods of the invention herein provide a fast and accurate test to identify isolates of S. aureus as MRSA, as confirmed by detection of the Mec motif and PVL plus or minus, based on detection of motifs corresponding to the phiSA2 prophage, which carries the PVL toxin gene (FIG. 9).

The embodiments of the disclosure may be carried out in other ways than those set forth herein without departing from the spirit and scope of the disclosure. The embodiments are, therefore, to be considered to be illustrative and not restrictive.

What is claimed is:

1. A method of determining antibiotic resistance, the method comprising the steps of:
(a) obtaining nucleic acid from a bacterium;
(b) imaging the nucleic acid;
(c) obtaining a restriction map of the nucleic acid;
(d) comparing the restriction map of the nucleic acid with a restriction map database; and
(e) determining antibiotic resistance of the bacterium by matching regions of said nucleic acid to corresponding regions in said database.

2. The method of claim 1, further comprising the step of linearizing said nucleic acid.

3. The method of claim 1, wherein the bacterium is at least one species selected from the group consisting of E. coli and S. aureus.

4. The method according to claim 3, wherein the S. aureus is a community-acquired methicillin-resistant strain of S. aureus.

5. The method according to claim 3, wherein the S. aureus is a hospital-acquired methicillin-resistant strain of S. aureus.

6. The method of claim 1, wherein the nucleic acid comprises substantially all genomic DNA of the bacterium.

7. The method of claim 1, wherein the nucleic acid comprises a transcriptome of the bacterium.

8. The method of claim 1, wherein the nucleic acid is deoxyribonucleic acid.

9. The method of claim 1, wherein the nucleic acid is ribonucleic acid.

10. The method of claim 1, further comprising digesting the nucleic acid with one or more enzymes prior to the imaging step.

11. The method of claim 10, wherein the enzymes are selected from the group consisting of: BglII, NcoI, XbaI, and BamHI.

12. The method of claim 1, wherein the imaging step comprises labeling the nucleic acid.

13. The method of claim 1, wherein the antibiotic is at least one selected from the group consisting of: methicillin, oxacillin, ciproflaxin, erythromycin, tetracyclin, clindamycin, trimethoprim/sulfa, vancomycin, penicillin G, levofloxacin, moxyfloxacin, rifampicin, linezolid, quinupristin/dalfopristin, gentamycin, and nitrofurantoin.

14. The method of claim 1, wherein the restriction database comprises a restriction map similarity cluster.

15. The method of claim 1, wherein the restriction database comprises a restriction map from at least one member of the clade of the organism.

16. The method of claim 1, wherein the restriction database comprises a restriction map from at least one subspecies of the organism.

17. The method of claim 31, wherein the restriction database comprises a restriction map from a genus, a species, a strain, a sub-strain, or an isolate of the organism.

18. The method of claim 1, wherein the database comprises a restriction map comprising motifs common to a genus, a species, a strain, a sub-strain, or an isolate of the organism.

19. The method of claim 18, wherein said motif is a mec cassette.

20. A method of determining an antibiotic resistance profile of a bacterium, the method comprising the steps of:
obtaining nucleic acid from a bacterium;
preparing an optical map of said nucleic acid;
identifying at least one motif present in said nucleic acid that is indicative of bacterial resistance; and
correlating said at least one motif with resistance to one or more antibiotics, thereby to determine the antibiotic resistance profile for said bacterium.

21. The method of claim 20, wherein said preparing step comprises linearizing said nucleic acid, digesting said nucleic acid with one or more restriction enzymes, and labeling said nucleic acid for imaging.

22. The method of claim 20, wherein said motif is selected from the group consisting of a mec cassette and psiSA2 prophage.

23. A method of determining a therapeutically effective antibiotic for treating a subject, the method comprising:
(a) obtaining a sample from a patient, wherein the sample is suspected to contain an infectious organism;
(b) obtaining a nucleic acid from the organism;
(b) preparing an optical map of said nucleic acid
(d) determining an antibiotic resistance profile of said organism by comparing said optical map with at least one database containing antibiotic resistance data; and
(e) selecting a therapeutically effective antibiotic for treating said patient.

24. The method of claim 23, further comprising the step of identifying said organism.

25. The method of claim 23, wherein the sample is selected from the group consisting of food, human tissue, body fluid, air, water, soil, plant material, and unknown material.

26. The method of claim 25, wherein the body fluid is selected from the group consisting of blood, sputum, saliva, urine, drainage from a body part, and drainage from an abscess.

27. The method of claim 23, wherein the organism is at least one type selected from the group consisting of a microorganism, a bacterium, a virus, and a fungus.

28. The method of claim 27, wherein the bacterium is at least one species selected from the group consisting of E. coli and S. aureus.

29. The method according to claim 28, wherein the S. aureus is a community-acquired methicillin-resistant strain of S. aureus.

30. The method according to claim 28, wherein the S. aureus is a hospital-acquired methicillin-resistant strain of S. aureus.

31. The method of claim 23, wherein said database comprises a restriction map similarity cluster.

32. The method of claim 23, wherein the restriction database comprises a restriction map from at least one member of the clade of the organism.

33. The method of claim 23, wherein the restriction database comprises a restriction map from at least one subspecies of the organism.

34. The method of claim 23, wherein the restriction database comprises a restriction map from a genus, a species, a strain, a sub-strain, or an isolate of the organism.

35. The method of claim 23, wherein the database comprises a restriction map comprising motifs common to a genus, a species, a strain, a sub-strain, or an isolate of the organism.
